(19) United States
(12) Patent Application Publication
(10) Pub. No.: US 2024/0257938 A1
(43) Pub. Date: Aug. 1, 2024

(54) HEALTH MANAGEMENT SYSTEM, HEALTH MANAGEMENT METHOD, AND COMPUTER READABLE MEDIUM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Takuhiro OMI, Anjo-shi (JP); Yuta MORI, Urayasu-shi (JP); Yasutomo MASUDA, Edogawa-ku (JP); Hajime KATSUDA, Nagareyama-shi (JP); Yumi KOYAMA, Shibuya-ku (JP)

(73) Assignee: TOYOTA JIDOSHA KABUSHIKI KAISHA, Toyota-shi (JP)

(21) Appl. No.: 18/424,330

(22) Filed: Jan. 26, 2024

(30) Foreign Application Priority Data
Jan. 31, 2023 (JP) 2023-012745

(51) Int. Cl. G16H 20/30; G16H 50/70

(52) U.S. Cl. CPC G16H 20/30; G16H 50/70

(57) ABSTRACT

A health management system comprising a first body shape prediction means for predicting a future body shape of a first user, a first predicted body shape image generation means for generating a first predicted body shape image showing the body shape predicted by the first body shape prediction means, a second body shape prediction means for predicting a future body shape of a second user, a second predicted body shape image generation means for generating a second predicted body shape image showing the body shape predicted by the second body shape prediction means, and output means for outputting the first predicted body shape image and the second predicted body shape image to a user terminal of the first user in such a way that both the first predicted body shape image and the second predicted body shape image are displayed, is provided.

### CROSS REFERENCE TO RELATED APPLICATIONS

This application is based upon and claims the benefit of priority from Japanese patent application No. 2023-012745, filed on Jan. 31, 2023, the disclosure of which is incorporated herein in its entirety by reference.

### BACKGROUND

The present disclosure relates to a health management system, a health management method, and a computer readable medium.

Patent Literature 1 (Japanese Unexamined Patent Application Publication No. 2014-147566) discloses a body shape change prediction apparatus for predicting a future change in a body shape of a user. Specifically, the body shape change prediction apparatus is configured to predict an increase or decrease in the body fat after a predicted period of time based on basic information such as age, sex, height, and weight of a target person whose change in the body shape is predicted, the body fat percentage, information regarding calorie intake per day, information regarding daily exercise, and a prediction period, process an image of the target person based on the results of the prediction, and output the processed image.

Patent Literature 2 (Japanese Unexamined Patent Application Publication No. 2017-91586) discloses a health management server that transmits a current appearance of a user whose health is managed and an expected future appearance of this user to a user terminal. The health management server determines a rival of a person whose health is managed and displays and outputs information on the appearance sent to the rival onto the user terminal.

### SUMMARY

In the above Patent Literature 1 and 2, there is a room for improving a motivation for managing health.

An object of the present disclosure is to provide a technique for improving a motivation for managing health.

According to a first aspect of the present disclosure, a health management system including: a first body shape prediction means for predicting a future body shape of a first user; a first predicted body shape image generation means for generating a first predicted body shape image showing the body shape predicted by the first body shape prediction means; a second body shape prediction means for predicting a future body shape of a second user; a second predicted body shape image generation means for generating a second predicted body shape image showing the body shape predicted by the second body shape prediction means; and output means for outputting the first predicted body shape image and the second predicted body shape image to a user terminal of the first user in such a way that both the first predicted body shape image and the second predicted body shape image are displayed is provided.

The future body shape may be a body shape obtained a predicted period of time after the present time.

The health management system may further include: a first current body shape image generation means for generating a first current body shape image showing a current body shape of the first user; and a second current body shape image generation means for generating a second current body shape image showing a current body shape of the second user, in which the output means outputs the first current body shape image, the second current body shape image, the first predicted body shape image, and the second predicted body shape image to the user terminal of the first user in such a way that the first current body shape image and the second current body shape image are displayed along with the first predicted body shape image and the second predicted body shape image.

The health management system may further include rival determination means for determining the second user among a plurality of users, in which the rival determination means may determine one of a plurality of users whose current body shape is the closest to the current body shape of the first user as the second user.

The health management system may further include rival determination means for determining the second user among a plurality of users, in which the rival determination means may determine one of a plurality of users whose target body shape is the closest to the target body shape of the first user as the second user.

The health management system may further include rival determination means for determining the second user among a plurality of users, in which the rival determination means may determine one of a plurality of users whose predicted body shape is the closest to the target body shape as the second user.

According to a second aspect of the present disclosure, a health management method causing a computer to execute: a first body shape prediction step of predicting a future body shape of a first user; a first predicted body shape image generation step of generating a first predicted body shape image showing the body shape predicted in the first body shape prediction step; a second body shape prediction step of predicting a future body shape of a second user; a second predicted body shape image generation step of generating a second predicted body shape image showing the body shape predicted in the second body shape prediction step; and an output step of outputting the first predicted body shape image and the second predicted body shape image to a user terminal of the first user in such a way that both the first predicted body shape image and the second predicted body shape image are displayed is provided.

According to a third aspect of the present disclosure, a health management program causing a computer to execute: a first body shape prediction step of predicting a future body shape of a first user; a first predicted body shape image generation step of generating a first predicted body shape image showing the body shape predicted in the first body shape prediction step; a second body shape prediction step of predicting a future body shape of a second user; a second predicted body shape image generation step of generating a second predicted body shape image showing the body shape predicted in the second body shape prediction step; and an output step of outputting the first predicted body shape image and the second predicted body shape image to a user terminal of the first user in such a way that both the first predicted body shape image and the second predicted body shape image are displayed is provided.

According to the present disclosure, it is possible to improve a motivation for managing health.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a schematic diagram of a health management system;

FIG. 2 is a functional block diagram of a health management server;

FIG. 3 is a functional block diagram of UE;

FIG. 4 is a diagram showing a display example of the UE; and

FIG. 5 is a control flow of the health management server.

### DESCRIPTION OF EMBODIMENTS

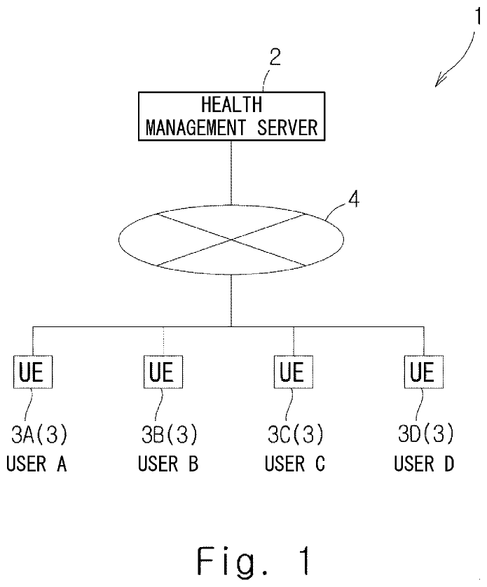

Hereinafter, with reference to FIGS. 1 to 5, an embodiment of the present disclosure will be described. FIG. 1 shows a schematic view of a health management system 1. As shown in FIG. 1, the health management system 1 includes a health management server 2 and a plurality of User Equipment (UE) 3. The health management server 2 and the plurality of UE 3 are configured in such a way that they can communicate with each other via a Wide Area Network (WAN) 4 such as the Internet. The plurality of UE 3 include UE 3A held by a user A, UE 3B held by a user B, UE 3C held by a user C, and UE 3D held by a user D. Each UE 3 is typically a mobile phone, a smartphone, a tablet, or a personal computer.

### (Health Management Server 2)

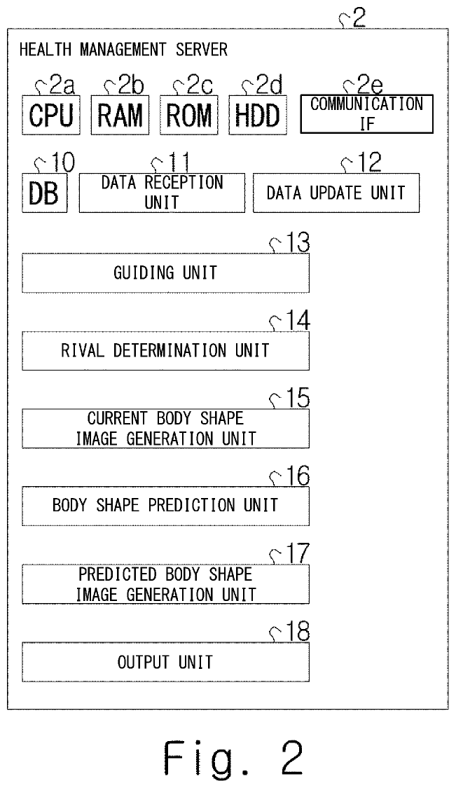

FIG. 2 shows a functional block diagram of the health management server 2. The health management server 2 provides a health management service for a plurality of users. The health management server 2 includes a Central Processing Unit (CPU) 2a, a readable/writable Random Access Memory (RAM) 2b, a read-only Read Only Memory (ROM) 2c. The health management server 2 further includes a Hard Disc Drive (HDD) 2d and a communication interface 2e. Then, the CPU 2a loads a control program stored in the ROM 2c or the HDD 2d and executes the loaded control program, whereby the control program causes hardware such as the CPU 2a to function as a database 10, a data reception unit 11, a data update unit 12, a guiding unit 13, a rival determination unit 14, a current body shape image generation unit 15, a body shape prediction unit 16, a predicted body shape image generation unit 17, and an output unit 18.

A plurality of user accounts are registered in the health management server 2.

The database 10 stores a user account, body shape data, exercise data, dietary data, target body shape data, and a guidance message in association with one another.

The body shape data is data indicating the current body shape of the corresponding user. The body shape typically includes height, weight, Body Mass Index (BMI), chest circumference, waist circumference, and other physical characteristics.

The exercise data is data indicating an exercise history of the corresponding user. The exercise history typically includes the date and time of exercise, a type of exercise, duration of exercise, and an amount of calories consumed.

The dietary data is data indicating a dietary history of the corresponding user. The dietary history typically includes the date and time of eating and calorie intake.

The target body shape data is data indicating a target body shape of the corresponding user.

The guidance message is a message indicating an exercise habit or a dietary habit that is necessary for the corresponding user to obtain the target body shape. The exercise habit is typically expressed by a daily exercise amount. The dietary habit is typically expressed by a daily meal amount.

The data reception unit 11 receives the body shape data, the exercise data, the dietary data, and the target body shape data from the UE 3 via the communication interface 2e.

The data update unit 12 updates the database 10 with the data received by the data reception unit 11.

The guiding unit 13 generates, for each user, a guidance message based on the exercise data, the dietary data, and the target body shape data. The guiding unit 13 updates the database 10 with the generated guidance message.

The rival determination unit 14 determines a user who will be a rival of the user A. Hereinafter, a user who will be a rival will be simply referred to as a rival.

For example, the rival determination unit 14 determines one of a plurality of users, except for the user A, whose current body shape is the closest to the current body shape of the user A as a rival. That is, by determining a user whose current body shape is similar to that of the user A as a rival, these users can, with regard to improving their body shapes, start from the same point.

Alternatively, the rival determination unit 14 may determine one of the plurality of users, except for the user A, whose target body shape is the closest to the target body shape of the user A as a rival. That is, by determining a user whose target body shape is similar to that of the user A as a rival, these users can, with regard to improving their body shapes, have the same goal.

Alternatively, the rival determination unit 14 may determine one of the plurality of users except for the user A whose predicted body shape is the closest to the target body shape as a rival. This is because a user with the smallest discrepancy between the predicted body shape and the target body shape may be a good example with regard to improving the body shape since such a user is highly motivated regarding health management.

The rival determination unit 14 determines rivals of other users in a way similar to that described above. That is, the rival determination unit 14 determines, for each user, the rival of this user.

The current body shape image generation unit 15 generates, for each user, the current body shape image showing the current body shape of this user based on the body shape data of this user. The current body shape image is typically an avatar image.

The body shape prediction unit 16 predicts, for each user, the body shape of this user after a predetermined period of time based on the exercise data and the dietary data of this user. “After a predetermined period of time” typically means after a predicted period of time from the present time. The predicted period is, for example, six months to 12 months.

The predicted body shape image generation unit 17 generates, for each user, a predicted body shape image showing the predicted body shape predicted by the body shape prediction unit 16. The predicted body shape image is generally an avatar image. The predicted body shape may be thinner than the current body shape if the user has been trying hard to lose weight or may be thicker than the current body shape if the user has been neglecting efforts to lose weight.

The output unit 18 outputs the current body shape images and the predicted body shape images of the two users who are rivals to the UE 3 they each have via the communication interface 2e so that these users can check each other's results regarding the improving of their body shapes.

Assume a case in which the user B is the rival of the user A. In this case, the output unit 18 outputs the current body shape image and the predicted body shape image of the user A and the current body shape image and the predicted body shape image of the user B to the UE 3A and the UE 3B. More specifically, the output unit 18 outputs, to the UE 3A and the UE 3B, the current body shape image and the predicted body shape image of the user A and the current body shape image and the predicted body shape image of the user B in such a way that both these images are displayed. At this time, the output unit 18 may integrate the current body shape image and the predicted body shape image of the user A with the current body shape image and the predicted body shape image of the user B to generate one image, and output the integrated image that has been generated to the UE 3A and the UE 3B.

### (UE 3)

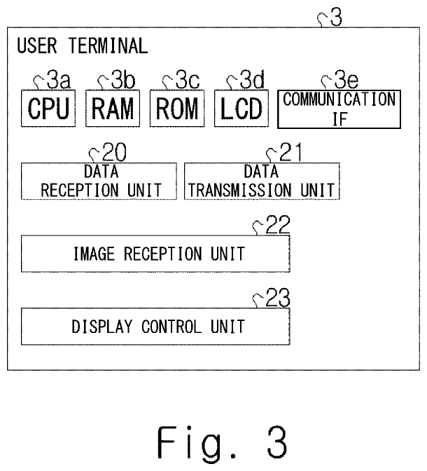

Next, with reference to FIG. 3, the UE 3 will be described. Hereinafter, the UE 3A held by the user A will be described. Further, it is assumed that the user B is the rival of the user A.

FIG. 3 shows a functional block diagram of the UE 3. The UE 3 is an information terminal held by the user who uses the health management server 2. The UE 3 includes a Central Processing Unit (CPU) 3a, a readable/writable Random Access Memory (RAM) 3b, and a read-only Read Only Memory (ROM) 3c. The UE 3 further includes a Liquid crystal display (LCD) 3d and a communication interface 3e. Then, the CPU 3a loads a control program stored in the ROM 3c and executes the loaded control program, whereby the control program causes hardware such as the CPU 3a to function as a data reception unit 20, a data transmission unit 21, an image reception unit 22, and a display control unit 23.

The data reception unit 20 receives data input by a user. The user inputs the body shape data, the exercise data, the dietary data, and the target body shape data to the UE 3.

The data transmission unit 21 transmits the body shape data, the exercise data, the dietary data, and the target body shape data input by the user to the health management server 2 via the communication interface 3e.

The image reception unit 22 receives the current body shape image and the predicted body shape image of the user A, and the current body shape image and the predicted body shape image of the user B from the health management server 2. Further, the UE 3 may receive a guidance message from the health management server 2.

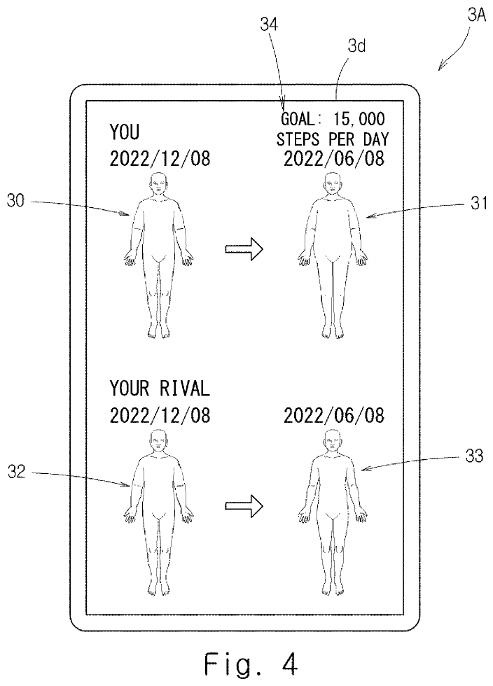

The display control unit 23 displays the current body shape image and the predicted body shape image of the user A and the current body shape image and the predicted body shape image of the user B received from the health management server 2 on the LCD 3d. FIG. 4 shows a display example on the LCD 3d of the UE 3A. As shown in FIG. 4, the LCD 3d of the UE 3A displays a current body shape image 30 and a predicted body shape image 31 of the user A, and a current body shape image 32 and a predicted body shape image 33 of the user B along with each other, that is, at the same time. The display control unit 23 may instead display the current body shape image 30 and the predicted body shape image 31 of the user A alternately with each other on the LCD 3d, for example, every few seconds. Likewise, the display control unit 23 may display the current body shape image 32 and the predicted body shape image 33 of the user B alternately with each other on the LCD 3d, for example, every few seconds. The displaying of the current body shape image 30 and the predicted body shape image 31 of the user A and the current body shape image 32 and the predicted body shape image 33 of the user B includes a state in which the current body shape image 30 and the predicted body shape image 31 of the user A, and the current body shape image 32 and the predicted body shape image 33 of the user B can be concurrently or alternately displayed on one screen. As shown in the display example of FIG. 4, the rival of the user A whose body shape is similar to that of the user A at the present time succeeds in losing weight after six months, while on the other hand, the user A who sees the results of the health management by the rival will clearly recognize that he/she has gained weight after six months, let alone losing weight. As a result, after reviewing his/her health management, the user A is likely to be highly motivated to improve the managing of his/her health.

As shown in FIG. 4, the display control unit 23 may cause the LCD 3d to display a guidance message 34 received from the health management server 2.

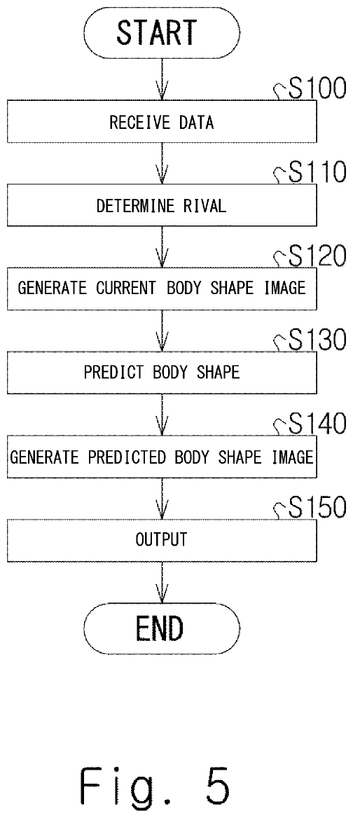

Next, with reference to FIG. 5, a control flow of the health management server 2 will be described.

First, the data reception unit 11 receives the body shape data, the exercise data, the dietary data, and the target body shape data from the UE 3 via the communication interface 2e (S100). The data update unit 12 updates the database 10 with the data received by the data reception unit 11.

Next, the rival determination unit 14 determines the rival for each user (S110).

Next, the current body shape image generation unit 15 generates, for each user, the current body shape image showing the current body shape of this user based on the body shape data of this user (S120).

Next, the body shape prediction unit 16 predicts, for each user, a body shape of the user after a predetermined period based on the exercise data and the dietary data of the user (S130).

Next, the predicted body shape image generation unit 17 generates, for each user, a predicted body shape image indicating the body shape predicted by the body shape prediction unit 16 (S140).

Next, the output unit 18 outputs the current body shape image and the predicted body shape image of the user A, and the current body shape image and the predicted body shape image of the user B to the UE 3A and the UE 3B via the communication interface 2e in such a way that both these images are displayed (S150).

While the embodiment of the present disclosure has been described above, the above embodiment has the following features.

That is, the health management server 2 includes the body shape prediction unit 16, the predicted body shape image generation unit 17, and the output unit 18. The body shape prediction unit 16 predicts a future body shape of the user A (a first user). The body shape prediction unit 16 predicts a future body shape of the user B (a second user). The body shape prediction unit 16 is one specific example of first body shape prediction means and second body shape prediction means. The predicted body shape image generation unit 17 generates the predicted body shape image 31 showing the future body shape of the user A predicted by the body shape prediction unit 16. The predicted body shape image generation unit 17 generates the predicted body shape image 33 showing the future body shape of the user B predicted by the body shape prediction unit 16. The predicted body shape image generation unit 17 is one specific example of first predicted body shape image generation means and second predicted body shape image generation means. The output unit 18 outputs the predicted body shape image 31 and the predicted body shape image 33 to the UE 3A of the user A in such a way that the predicted body shape image 31 of the user A (a first predicted body shape image) and the predicted body shape image 33 of the user B (a second predicted body shape image) are both displayed. According to the aforementioned configuration, it is possible to improve the motivation of the user A for managing health.

The aforementioned future body shape is a body shape obtained a predicted period of time after the present time. The predicted period of time is, for example, six months or 12 months. According to the aforementioned configuration, the body shape prediction unit 16 is able to predict long-term outcomes by health management.

The health management server 2 further includes the current body shape image generation unit 15. The current body shape image generation unit 15 generates the current body shape image 30 showing the current body shape of the user A (a first current body shape image). The current body shape image generation unit 15 generates the current body shape image 32 showing the current body shape of the user B (a second current body shape image). The current body shape image generation unit 15 is one specific example of first current body shape image generation means and second current body shape image generation means. Then, as shown in FIG. 4, the output unit 18 outputs the current body shape image 30, the current body shape image 32, the predicted body shape image 31, and the predicted body shape image 33 to the UE 3A of the user A in such a way that the current body shape image 30 and the current body shape image 32 are displayed along with the predicted body shape image 31 and the predicted body shape image 33. According to the aforementioned configuration, the user A can compare the current body shape image 30 with the predicted body shape image 31, compare the current body shape image 32 with the predicted body shape image 33, compare the current body shape image 30 with the current body shape image 32, and compare the predicted body shape image 31 with the predicted body shape image 33. Accordingly, the user A can easily recognize the difference between the motivation of the user A for managing health and the motivation of the user B for managing health. That is, in the example shown in FIG. 4, the user A will recognize that his/her motivation for managing health is much lower than the motivation of the user B for managing health. Then, the user A will increase his/her motivation for managing health under the influence of the user B.

The above embodiment may be changed, for example, as follows. That is, the rival determination unit 14 may determine a user X, who is a user A at a past first time point, as a rival of the user A. In this case, the current body shape image generation unit 15 generates a current body shape image of the user X based on body shape data of the user X. The predicted body shape image generation unit 17 generates a predicted body shape image of the user X based on body shape data of the user X at a second time point, which is after an elapse of a predicted period of time from the first time point. Then, the output unit 18 outputs the current body shape image and the predicted body shape image of the user A and the current body shape image and the predicted body shape image of the user X to the UE 3A in such a way that these images are all displayed. In this manner, the user A can believe that his/her health management efforts will be rewarded by regarding his/her own past success as a rival, whereby it will be possible to improve his/her motivation for managing health.

A (The) program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A health management system comprising:
at least one memory acquiring storing instructions and
at least one processor configured to execute the instructions to;
predict a future body shape of a first user;
generate a first predicted body shape image showing the body shape predicted by the first body shape prediction means;
predict a future body shape of a second user;
generate a second predicted body shape image showing the body shape predicted by the second body shape prediction means; and
output the first predicted body shape image and the second predicted body shape image to a user terminal of the first user in such a way that both the first predicted body shape image and the second predicted body shape image are displayed.

2. The health management system according to claim 1, wherein the future body shape is a body shape obtained a predicted period of time after the present time.

3. The health management system according to claim 1, wherein the at least one processor is further configured to execute the instructions to:
generate a first current body shape image showing a current body shape of the first user;
generate a second current body shape image showing a current body shape of the second user, and
output the first current body shape image, the second current body shape image, the first predicted body shape image, and the second predicted body shape image to the user terminal of the first user in such a way that the first current body shape image and the second current body shape image are displayed along with the first predicted body shape image and the second predicted body shape image.

4. The health management system according to claim 1, wherein the at least one processor is further configured to execute the instructions to:
determine the second user among a plurality of users, and
determine one of a plurality of users whose current body shape is the closest to the current body shape of the first user as the second user.

5. The health management system according to claim 1, wherein the at least one processor is further configured to execute the instructions to:
determine the second user among a plurality of users, and
determine one of a plurality of users whose target body shape is the closest to the target body shape of the first user as the second user.

6. The health management system according to claim 1, wherein the at least one processor is further configured to execute the instructions to:
determine the second user among a plurality of users, and
determine one of a plurality of users whose predicted body shape is the closest to the target body shape as the second user.

7. A health management method causing a computer to execute:
a first body shape prediction step of predicting a future body shape of a first user;
a first predicted body shape image generation step of generating a first predicted body shape image showing the body shape predicted in the first body shape prediction step;
a second body shape prediction step of predicting a future body shape of a second user;
a second predicted body shape image generation step of generating a second predicted body shape image showing the body shape predicted in the second body shape prediction step; and
an output step of outputting the first predicted body shape image and the second predicted body shape image to a user terminal of the first user in such a way that both the first predicted body shape image and the second predicted body shape image are displayed.

8. A non-transitory computer readable medium storing a health management program for causing a computer to execute:
a first body shape prediction step of predicting a future body shape of a first user;
a first predicted body shape image generation step of generating a first predicted body shape image showing the body shape predicted in the first body shape prediction step;
a second body shape prediction step of predicting a future body shape of a second user;
a second predicted body shape image generation step of generating a second predicted body shape image showing the body shape predicted in the second body shape prediction step; and
an output step of outputting the first predicted body shape image and the second predicted body shape image to a user terminal of the first user in such a way that both the first predicted body shape image and the second predicted body shape image are displayed.
